(19) United States
(12) Patent Application Publication
(10) Pub. No.: US 2004/0230175 A1
(43) Pub. Date: Nov. 18, 2004

(54) Re-usable and washable protective panty

(76) Inventors: Francine Rainville-Lonn, Cornwall (CA); Harvey Lonn, Cornwall (CA)

Correspondence Address: OGILVY RENAULT, 1981 MCGILL COLLEGE AVENUE, SUITE 1600, MONTREAL, QC, H3A2Y3, CA

(21) Appl. No.: 10/439,230

(22) Filed: May 16, 2003

(51) Int. Cl. A61F 13/15; A61F 13/20

(52) U.S. Cl. 604/396; 604/385.01

(57) ABSTRACT

A re-usable and washable protective panty for wear by an incontinent person is described. The panty comprises an outer elastic fabric cut and stitched to define a waist opening and leg openings. The panty defines a front panel and a rear panel. The rear panel has a seat area. A crotch region is defined between the front and rear panels and extends between the leg openings. A non-breathable, non-stretchable, waterproof polyester panel is permanently secured over an inner surface of the panty and spans the crotch region. The waterproof polyester panel is stitched along a peripheral edge thereof to the inner surface of the panty and is free of seams spanning the peripheral edge in the crotch region. The waterproof polyester panel has a contour shape to substantially shield the outer elastic fabric from body fluids from a wearer person when in an upright, sitting or lying position while retaining a substantially flat shape in the crotch region.

### TECHNICAL FIELD

The present invention relates to a re-usable and washable protective panty for wear by an incontinent person.

### BACKGROUND ART

Sanitary panty garments having a special crotch section to protect a wearer in that area are known. For example, U.S. Pat. No. 4,813,950 describes such a sanitary panty to prevent leakage and soiling of outer garments. Such a panty is also constructed to be substantially unaffected by repeated washing and drying without the need for special care. In order to provide protection, plastic film inner layers extend through the crotch region and are secured permanently thereto by stitching whereby to conform to the shape of the outer material covering. It has been found that this type of construction results in kinking and canalization of the protective material and causes leakage to take place along the edges of the leg openings of the panty as the protection is not substantially uniform throughout the crotch region. These panties are also not comfortable to the wearer person.

U.S. Pat. No. 5,098,419 also describes another example of such undergarment.

### SUMMARY OF INVENTION

It is a feature of the present invention to provide a re-usable and washable protective panty for wear by an incontinent person and which is an improvement over the known prior art described hereinabove.

Another feature of the present invention is to provide a re-usable and washable protective panty for wear by an incontinent person and wherein a waterproof polyester panel is shaped and stitched such as to substantially shield the outer elastic fabric from body fluids from a wearer person when in an upright, sitting or lying position, whether lying on one's back or front and which is capable of retaining a substantially flat shape in the crotch region.

Another feature of the present invention is to provide a re-usable and washable liner pad in combination with the protective panty.

According to the above features, from a broad aspect, the present invention provides a re-usable and washable protective panty for wear by an incontinent person. The protective panty comprises an outer elastic fabric cut and stitched to define a waist opening and leg openings. The panty defines a front panel and a rear panel. The rear panel has a seat area. A crotch region is defined between the front and rear panels and extends between the leg openings. A non-breathable, non-stretchable, waterproof polyester panel is permanently secured over an inner surface of the panty and spans the crotch region. The waterproof polyester panel is stitched along a peripheral edge thereof to the inner surface of the panty and is free of seams spanning the peripheral edge in the crotch region. The waterproof polyester panel has a contour shape to substantially shield the outer elastic fabric from body fluids from a wearer person when in an upright, sitting or lying position while retaining a substantially flat shape in the crotch region.

### BRIEF DESCRIPTION OF DRAWINGS

A preferred embodiment of the present invention will now be described with reference to the accompanying drawings in which:

FIG. 1 is a front elevation view of the re-usable and washable protective brief panty construction in accordance with the present invention;

FIG. 2 is a front elevation view, partly fragmented, of a re-usable and washable protective bikini panty constructed in accordance with the present invention;

FIG. 3 is a side view of FIG. 1;

FIG. 4 is a fragmented view of the panty showing the waterproof polyester panel permanently secured on an inner surface of the crotch region of the panty;

FIG. 5A is a view similar to FIG. 4 but showing retention panels secured along opposed top edges of the waterproof polyester panel to retain a liner pad; and

FIG. 5B is a perspective view showing a removable, re-usable and washable panty liner pad removably secured over the outer surface of the waterproof polyester panel.

### DESCRIPTION OF PREFERRED EMBODIMENTS

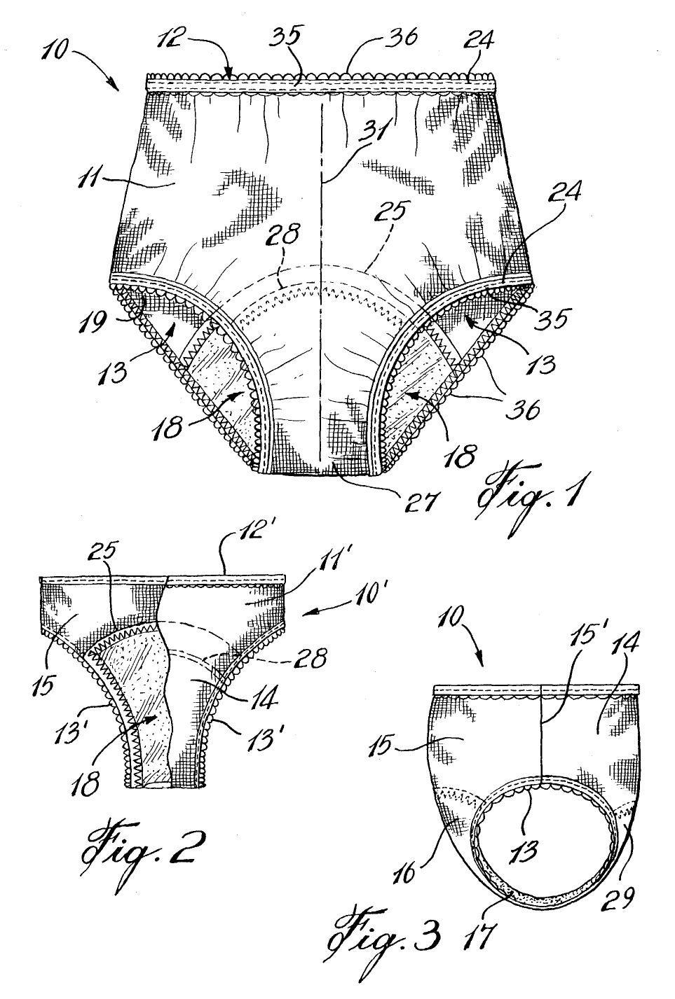

Referring now to FIGS. 1 to 3, there is shown in FIG. 1, a re-usable and washable protective brief-type panty 10 constructed in accordance with the present invention. FIG. 2 shows the protective panty as being of a brief type 10′ and constructed in substantially the same manner as illustrated in FIG. 1. The protective panties 10 and 10′ comprise an outer elastic fabric 11 and 11′, respectively which is cut and stitched to define a waist opening 12 and 12′ and leg openings 13 and 13′, respectively. As illustrated more clearly in FIG. 3, the panty defines a front panel 14 and a rear panel 15 stitched together by vertical seams 15 on opposed sides thereof. The rear panel 15 defines a seat area 16. A crotch region 17 is defined between the front and rear panels and extends between the leg openings 13.

As shown in FIGS. 1 and 2, a non-breathable, non-stretchable, waterproof polyester panel 18 is permanently secured over an inner surface 19 of the front and rear panels 14 and 15. As can be seen, this waterproof polyester panel 18 spans the crotch region 17.

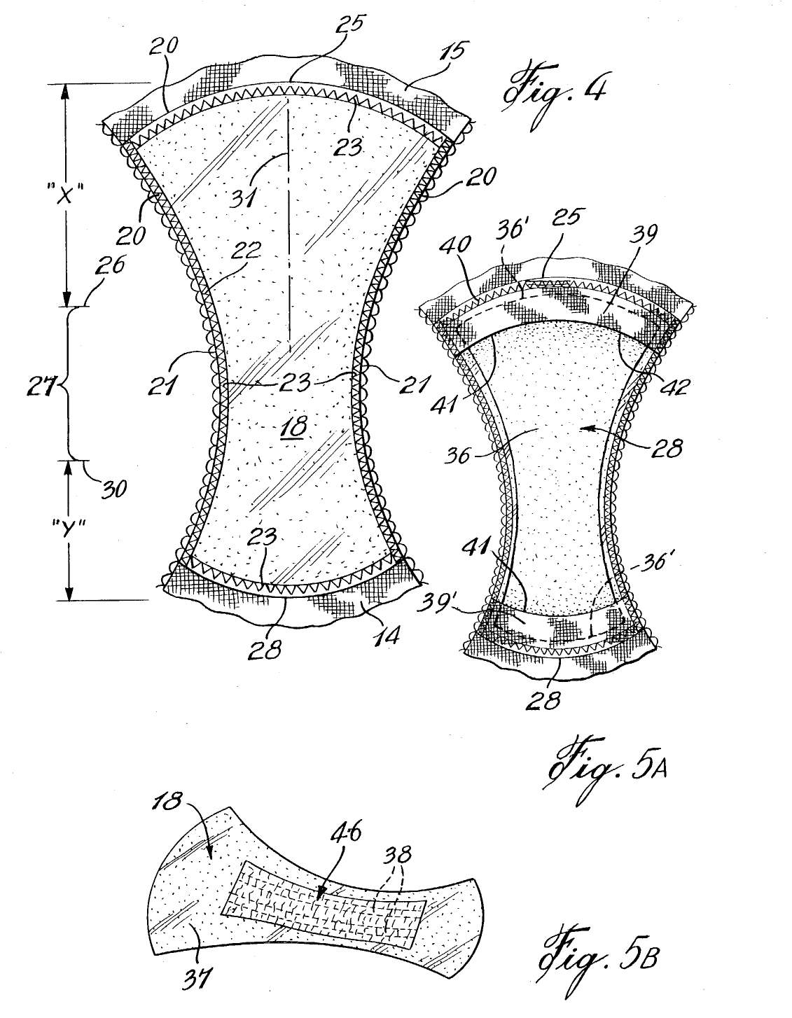

With reference now to FIG. 4, it can be seen that the waterproof polyester panel 18 is stitched along a peripheral edge 20 thereof to the inner surface 19 of the panels 14 and 15 and is also free of seams spanning the peripheral edge in the crotch region. The waterproof polyester panel has a contour shape to substantially shield the outer elastic fabric panels 14 and 15 from body fluids from a wearer person whether the wearer person is upright, sitting or in a lying position, either on one's back or front, with the panel retaining a substantially flat shape in the crotch region.

The shape of the waterproof polyester panel defines opposed concave side edges 21 which are stitched along a respective one of the leg openings 13 by a poly core thread 22 defining a zig-zag 3-step stitch 23. An outer cover stitch 24, see FIG. 1, extends over the outer surface of the outer fabric along the zig-zag stitch 23. The waterproof polyester panel 18 also defines a large convex top rear edge 25 which delineates the top end of the seat section or seat area 16, defined from a rear edge 26 of a central region 27 of the panel 15. A smaller convex top front edge 28 defines a frontal section 29, see FIG. 3, which extends from a front edge 30 of the central region 27. As shown in FIG. 4, the distance “x” from the rear edge 26 of the central region 27 along a central longitudinal axis 31 of the waterproof polyester panel 18 to the convex top rear edge 25 is greater than the distance “y” between the front edge 30 of the central region 27 to the convex top front edge 28 of the frontal section 29 of the panel 18. The distance “x” of a particular embodiment of the panty is 4 inches while the distance “y” is 3½ inches. The central region has a length of about 5¼ inches along the longitudinal central axis 31.

It is pointed out that the front and rear panels 14 and 15, respectively, may be fabricated from various outer fabrics such as Jacquard, lace or plain nylon-spandex or cotton lycra.

As shown in FIG. 1, an elastic decorative band 35 with decorative edging 36 is sewn about the waist and leg openings with a poly-core thread forming the cover stitch 24.

Referring now to FIGS. 5A and 5B, there is illustrated the use of a removable, re-usable and washable panty liner pad 46 used in combination with the waterproof polyester panel 18 whereby to provide a complement to sanitary pads or tampons and to incontinence pads or for light incontinence such as during pregnancy, when a woman sneezes, coughs or during vaginal discharge. This liner pad 46 may be comprised of two or more plies of soaker material and polyester material quilted together. As shown in FIG. 5B, this liner pad 46 may have a different shape and may be adhered to the outer surface 37 of the waterproof polyester panel 18 by such means as adhesive strips 38, or adhesive wings formed integral with the pads and not illustrated herein but well known in the art. As shown in FIG. 5A, liner pad retention panels 39, 39′ are formed of waterproof and washable polyester material and are secured at opposed ends of the polyester panel 18 adjacent the convex top rear edge 25 and the top convex front edge 28. These liner pad retention panels are secured by zig-zag stitches 40 disposed along a top edge and opposed side edges thereof. A front edge 41 defines a mouth opening 42 to receive opposed end portions 36′ of the liner pad 46.

It is pointed out that this washable and re-usable protective panty can be worn days and nights for complete protection against staining on clothing and bed linens as well as articles of furniture. It eliminates the worry of a person with incontinence as it provides additional and effective continuous protection regardless of the activity or position of the wearer. Because there are no seams in the crotch region of the waterproof polyester panel, it remains more flat to provide even protection throughout, regardless of the position of the wearer. The decorative edging along the waist and leg openings is disposed on the outside of the panty and therefore is not stained by liquid discharge from the wearer. The protective waterproof panel also does not retain odors and it can be wiped clean with a wet paper towel or cloth and can be wiped dry using a dry tissue or cloth. Although this protective panel 18 is non-breathable, it will not irritate or shafe the skin of the wearer and will not create moisture. Although this panel does not stretch, the panty is created in a way that it allows the panel to take the form of the body. The panty is also machine wash-and-dry and is further bleachable.

The added feature of being able to secure a panty liner pad to the waterproof polyester panel makes the panty use more economical and environmentally friendly. It is the perfect compliment to disposable pads and tampons. The panty liner pad could also be made from one ply dry force polyester and one or more ply of soaker quilted together and laminated to a one-ply material known as Comfort to provide more protection and also to prevent a person from touching the elimination.

It is within the ambit of the present invention to cover any obvious modifications of the preferred embodiment described herein, provided such modifications fall within the scope of the appended claims.

We claim:

1. A re-usable and washable protective panty for wear by an incontinent person, said protective panty comprising an outer elastic fabric cut and stitched to define a waist opening and leg openings, said panty defining a front panel and a rear panel, said rear panel having a seat section, a crotch region between said front and rear panels and extending between said leg openings; a non-breathable non-stretchable, waterproof polyester panel permanently secured over an inner surface of said panty and spanning said crotch region; said waterproof polyester panel being stitched along a peripheral edge thereof to said inner surface of said panty and being free of seams spanning said peripheral edge in said crotch region, said waterproof polyester panel having a contour shape to substantially shield said outer elastic fabric from body fluids from a wearer person when in an upright, sitting or lying position while retaining a substantially flat shape in said crotch region.

2. A protective panty as claimed in claim 1 wherein said waterproof polyester panel contour shape has opposed concave side edges stitched along a respective one of said leg openings in said crotch region, a central region defined between said concave side edges, a large convex top rear edge delineates a top end of said seat section from a rear edge of said central region, and a smaller convex top front edge defining a frontal section from a front edge of said central region, the distance “x” from said rear edge of said central region along a central longitudinal axis of said waterproof polyester panel to said convex top rear edge being greater than the distance “y” between said front edge of said central region to said convex top front edge of said frontal section.

3. A protective panty as claimed in claim 2 wherein said distance “x” is 4 inches, said distance “y” is 3½ inches, said central region having a length of 5¼ inches along said central longitudinal axis.

4. A protective panty as claimed in claim 1 wherein said outer elastic fabric is one of Jacquard, lace or plain nylon-spandex, or cotton lycra.

5. A protective panty as claimed in claim 1 wherein said stitch along said peripheral edge of said waterproof polyester panel is comprised of a poly-core thread having a zig-zag three-step stitch, and an outer cover stitch over said zig-zag stitch along said leg openings.

6. A protective panty as claimed in claim 5 wherein an elastic band with a decorative edging is sewn about said waist and leg openings with a poly-core thread using a cover stitch.

7. A protective panty as claimed in claim 1 wherein said panty is one of a brief or bikini shaped panty.

8. A protective panty as claimed in claim 2 wherein a removable, re-usable and washable panty liner pad is removably securable to said waterproof polyester panel.

9. A protective panty as claimed in claim 8 wherein said liner pad is comprised of two or more plies of soaquer and polyester material quilted together, and a liner pad retention panel secured at opposed ends of said waterproof polyester panel adjacent said convex top rear edge and top front edge for receiving a respective end portion of said liner pad thereunder, said retention panels being zig-zag stitched along a top edge and opposed side edges thereof, said front edge defining a mouth opening.
